(19) United States
(12) Patent Application Publication
(10) Pub. No.: US 2005/0255448 A1
(43) Pub. Date: Nov. 17, 2005

(54) System for amplifying optical detection of cantilever deflection

(76) Inventors: Arun Majumdar, Orinda, CA (US); Srinath Satyanarayana, Berkeley, CA (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 10/883,964

(22) Filed: Jul. 6, 2004

Related U.S. Application Data
(60) Provisional application No. 60/571,484, filed on May 13, 2004.

(51) Int. Cl. C12Q 1/00; C12M 1/34

(52) U.S. Cl. 435/4; 435/287.1

(57) ABSTRACT

A sensor system configured to amplify cantilever deflection, having: a reflective membrane connected to a cantilever, wherein the reflective membrane rotates more than the cantilever when the cantilever deflects. A sensor system, including: a cantilever having a first end and a second end, the first end being held at a first fixed location and the second end being free to move; and a reflective membrane having a first end and a second end, the first end being held at a second fixed location and the second end being attached to the cantilever.

### PRIORITY CLAIM

The present application claims priority under 35 U.S.C. § 119 to U.S. Provisional Application No. 60/571,484, (attorney Docket number 028726-036), entitled Microcantilever And Micromembrane Systems Having Reaction Surfaces Configured For Molecule To Molecule, Molecule To Cell, Cell To Molecule And Cell To Cell Bonding, filed May 13, 2004.

### STATEMENT OF GOVERNMENT-SUPPORTED RESEARCH

This invention was made with Government support under Grant (Contract) No. R21 CA86132-01 awarded by the National Institutes of Health/National Cancer Institute and Contract No. DE-FG03-98ER14870 awarded by the United States Department of Energy. The Government has certain rights in this invention.

### TECHNICAL FIELD

The present invention relates generally to sensing systems, and in particular to microcantilever sensing systems. The present invention may be used for various cantilever sensing applications, including physical, chemical, and biological sensing.

### BACKGROUND

Micro- or nano-cantilevers have been used as sensors, for example as physical, chemical, and biological sensors. As commonly understood, and as used herein, the term cantilever refers to a structure that is fixed at one end and free at the other.

Some existing cantilever sensors are based on the principle that a change in surface stress, produced by interaction with the environment, results in a deflection of the free end of the cantilever. Such deflection of the free end of the cantilever results in rotation of portions of the surface of the cantilever.

The deflection and rotation of the cantilever may be measured by a number of different approaches. One method is referred to as the Optical Beam Deflection Method (OBDM). In this method, a laser beam is reflected off the cantilever, and the cantilever deflection/rotation is measured by movement of the reflected laser beam. Commonly, the incident laser beam is directed at the portion of the cantilever where the deflection/rotation is at a maximum (i.e: toward the free end of the cantilever). In various systems, the deflection/rotation of the cantilever is determined by detecting a change in position of the reflected beam (from a first cantilever) with respect to the position of the reflected beam from a control (i.e. second) cantilever. The reflected beam may be detected using, for example, a position sensitive detector (PSD) or a charge coupled device (CCD) camera.

An example of such an existing optical beam deflection detection is seen in FIG. 1, wherein the change in position of a reflected beam (d₁) is proportionate to the deflection (δ₁) and rotation (θ₁) of the cantilever. For example, when the cantilever is not deflected (shown as cantilever 10A), the incident beam from laser 12 will be deflected along path (d₁A) toward detector array 14. However, after the cantilever has been deflected (shown as cantilever 10B), the incident beam will instead be deflected along path (d₁B) toward detector array 14. Unfortunately, the resolution of this method of detection is limited by the resolution of the optical detection system (i.e.: the resolution of detector array 14). Specifically, small cantilever deflections yield only small differences between beam paths d1A and d1B. Therefore, it is difficult to detect small cantilever deflections. This may be undesirable since certain reactions to be detected may only produce small detections.

Microcantilevers have been used to sense biomolecular interactions, as follows. In order to identify particular biological molecules, a probe molecule is disposed on the microcantilever, wherein the probe molecule interacts with the particular biological molecule to be detected. For example, in order to detect particular DNA material, a short single-stranded DNA (ssDNA) sequence may be used as a probe molecule for a complimentary ssDNA. Similarly, in order to detect a particular antigen, an appropriate antibody may used as a probe molecule.

The presence of the particular biological molecule (i.e.: the “target molecule”) may be detected by first functionalizing a surface of the cantilever using an appropriate probe molecule (i.e.: attaching probe molecules to the surface of the cantilever), and then detecting a resulting physical change in the cantilever. When a target molecule binds to one of the probe molecules on the surface of the cantilever, the cantilever bends due to a change in its surface stress. Determining the amount by which the cantilever bends provides a measure of the concentration of the molecule to be detected.

### SUMMARY

The present invention provides a system for amplifying optical detection of cantilever deflection. In a preferred embodiment, a reflective membrane is attached to the cantilever such that the reflective membrane rotates more than the cantilever when the cantilever deflects. In preferred embodiments, an incident beam is reflected off of the reflective membrane (instead of the cantilever). Since the reflective membrane rotates more than the cantilever, a larger deflection of the beam is detected.

In a preferred embodiment, the present invention provides a sensor system, having: a cantilever having a first end and a second end, the first end being held at a first fixed location and the second end being free to move; and a reflective membrane having a first end and a second end, the first end being held at a second fixed location and the second end being attached to the cantilever.

In optional preferred embodiments, the cantilever is attached directly to a wall and the reflective membrane is attached to a substrate that is in turn attached to the wall.

The details of one or more implementations are set forth in the accompanying drawings and the description below. Other features and advantages will be apparent from the description and drawings, and from the claims.

### DESCRIPTION OF DRAWINGS

FIG. 1 shows a prior art method of optical detection of cantilever deflection.

FIG. 2A is a side elevation view of an embodiment of the present invention, prior to cantilever deflection (and membrane rotation).

FIG. 2B is a side elevation view corresponding to FIG. 2A, after cantilever deflection (and membrane rotation).

FIG. 2C is a top plan view corresponding to FIG. 2A.

FIG. 3 shows a top plan view of another embodiment of the invention including a double cantilever, a pair of membranes, and a reflective panel across the pair of membranes.

FIG. 4A is a side elevation view off a prior art cantilever (i.e: without the present reflective membrane attached thereto), showing cantilever deflection.

FIG. 4B is a side elevation view showing the present cantilever (i.e.: with the reflective membrane attached to the end of the cantilever), showing cantilever deflection and membrane rotation.

FIG. 4C shows a free-body diagram for a structure including a cantilever and membrane corresponding to FIG. 4B.

FIG. 5 is a graph of mechanical amplification as a function of membrane length for five different cantilever and reflective membrane embodiments of the invention.

Like reference symbols in the various drawings indicate like elements.

### DETAILED DESCRIPTION

The present invention provides a system for amplifying micro- or nano-cantilever deflection for optical detection. It is to be understood, however, that the present invention may also be used to amplify deflections of various membrane sensors.

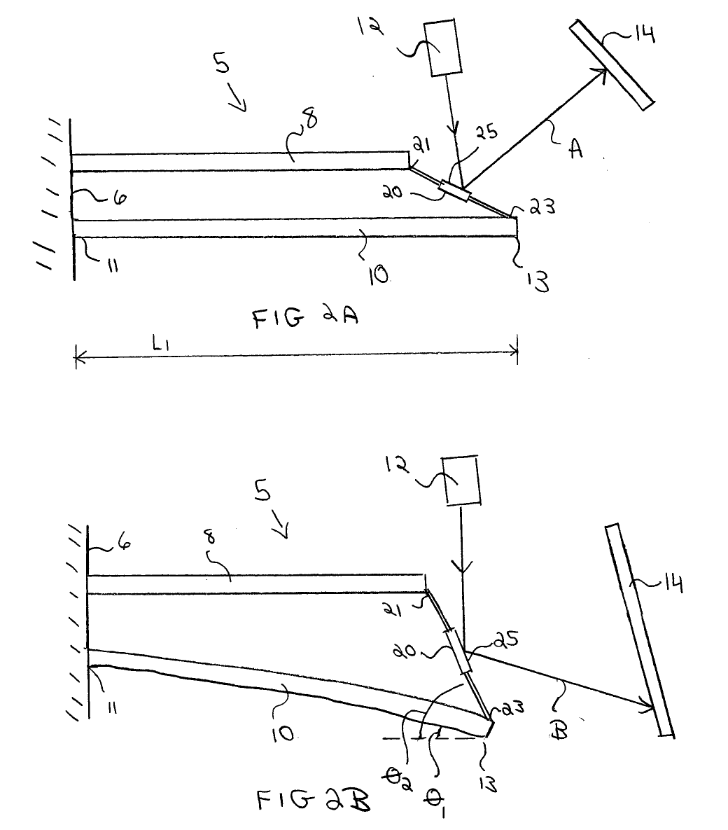

A preferred embodiment of the present invention is illustrated in FIGS. 2A and 2B in which a single reflective membrane is attached to a single cantilever. This embodiment includes a single cantilever and a single membrane. As will be shown, however, the present invention may also include systems with two or more reflective membranes attached to a one or more cantilevers.

As illustrated in FIG. 2A, the present invention provides a sensor system 5, including a cantilever 10 having a first end 11 and a second end 13. The first end 13 is held at a first fixed location and the second end 13 is free to move (i.e.: deflect up/down). A reflective membrane 20 having a first end 21 and a second end 23 is provided. The first end 21 is held at a second fixed location and the second end 23 is attached to cantilever 10 at its end free 13. The entire surface of membrane 20 may be reflective, or alternately, only a reflective panel (or paddle) portion 25 need be reflective.

As illustrated, first end 21 of reflective membrane 20 is attached to a stationary substrate 8. As is seen in FIGS. 2A and 2B, cantilever 10 and substrate 8 may both be attached to a wall 6. In preferred embodiments, substrate 8 and cantilever 10 may be generally parallel to one another prior to deflection of cantilever 10, as shown.

In operation, a beam of laser light from laser 12 is reflected from reflecting membrane 20 towards detector array 14. Prior to cantilever deflection, as shown in FIG. 2A, the reflected light is directed along path “A”. However, after cantilever deflection, as shown in FIG. 2B, the reflected light is instead directed along path “B”.

As can be seen in FIG. 2B, when cantilever 10 deflects, (e.g.: when free end 13 of cantilever 10 moves downwardly), the reflective surface of membrane 20 rotates such that the beam of laser light is directed along path “B” towards detector array 14.

Therefore, deflection of cantilever 10 causes rotation of the reflective membrane 20. Advantageously, the angle to which reflective membrane 20 rotates is greater than the angle to which the portion of cantilever 10 to which second end 23 of reflective membrane 20 is attached rotates. As can be seen in both FIGS. 2A and 2B, cantilever 10 is longer than reflective membrane 20.

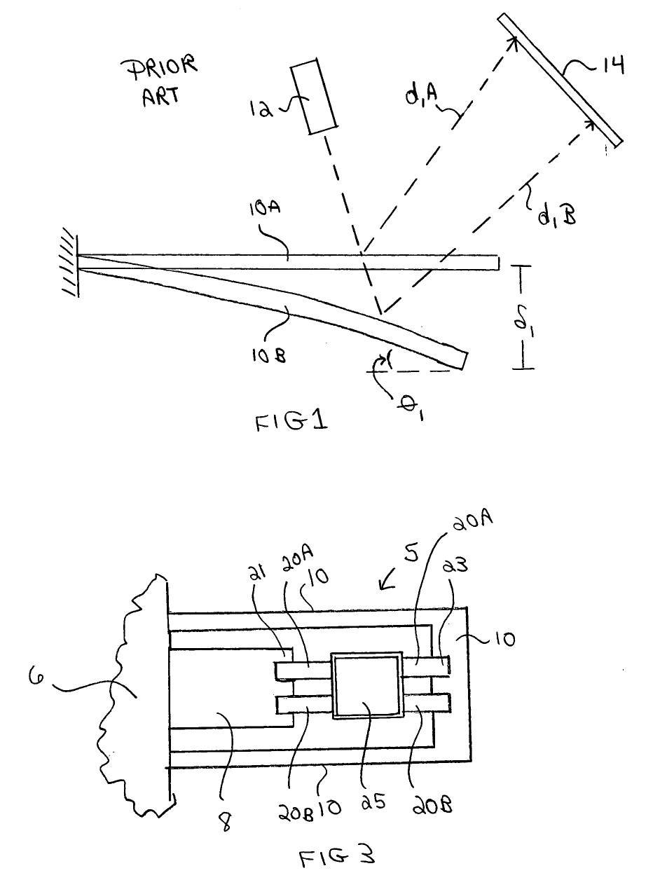

Thus, the angle of rotation of reflective membrane 20 is greater than the angle of rotation of second end 13 of the cantilever when cantilever 10 deflects. This is particularly advantageous in optical detection since the difference in the angle between light paths “A” and “B” in FIGS. 2A and 2B is greater than the difference in the angle between light paths d1A and d1B in FIG. 1.

In preferred embodiments, reflective membrane 20 may comprise a thin flexible membrane that may optionally be made out of Parylene, but is not so limited. In various embodiments, reflective membrane 20 may comprise a membrane with a layer of reflective material coated or deposited thereon. Alternatively, reflective membrane 20 may comprise a membrane with a panel of reflective material attached thereto. Thus, reflective membrane 20 may have a first portion that is reflective, and a second portion that is not reflective.

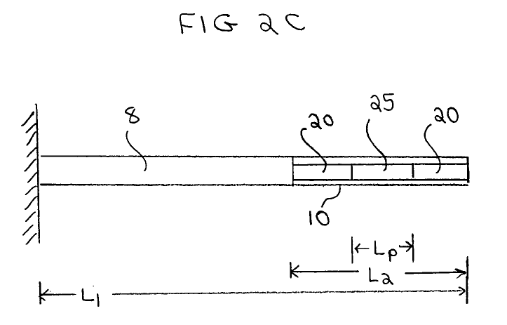

FIG. 3 illustrates a top plan view of an optional embodiment of the invention in which the reflecting membrane comprises a pair of membrane strips 20A and 20B, each having its first end 21 connected to a fixed substrate 8 and its second end 23 connected to cantilever 10. A panel 25 of reflective material is disposed across membrane strips 20A and 20B.

Cantilever 10 may be deflected by any of a variety of methods, including, but not limited to mechanical, chemical, electrical and magnetic systems. In one exemplary embodiment, cantilever 10 has probe molecules or cells disposed thereon, and cantilever 10 deflects in response to surface stress changes caused by target molecules or cells interacting with such probe molecules or cells. Specifically, when a target material interacts with one or more of the probe molecules, the surface stress of cantilever 10 changes, causing cantilever 10 to deflect. The target molecules or cells may be exposed to cantilever 10 by a sample fluid or gas surrounding cantilever 10.

The present invention also includes a method of sensing cantilever deflection, by directing a beam of incident light towards reflective membrane 20, wherein first end 21 of reflective membrane 20 is held at a fixed location and second end 23 of reflective membrane 20 is attached to cantilever 20; deflecting cantilever 20; and detecting movement of a beam of light reflected by reflective membrane 20.

In a preferred aspect of this method, detecting the movement of the beam of light reflected by the reflective membrane 20 corresponds to determining target molecule or cell concentration.

Various preferred geometries for the present invention will now be set forth. Table 1 below shows the nomenclature used herein.

TABLE 1

| | |
|---|---|
| L₁ | Length of cantilever 10 |
| w₁ | Width of cantilever 10 |
| t₁ | Thickness of cantilever 10 |
| I₁ | Bending moment of inertia of cantilever 10 = w₁t₁³/12 |
| A₁ | Cross-sectional area of cantilever 10 = w₁t₁ |
| E₁ | Elastic modulus of cantilever 10 |
| ν₁ | Poisson's ratio of cantilever 10 |
| L₂ | Length of combined membrane 20 and reflective portion 25 |
| L_{P} | Length of reflective portion 25 |
| L₃ | Length of membrane 20 = L₂ − Lₚ |
| w₂ | Width of membrane 20 |
| t₂ | Thickness of membrane 20 |
| I₂ | Bending moment of inertia of membrane 20 = w₂t₂³/12 |
| A₂ | Cross-section area of membrane 20 = w₂t₂ |
| E₂ | Elastic modulus of membrane 20 |
| ν₂ | Poisson's ratio of membrane 20 |
| γ | Surface stress induced in cantilever 10 (N/m) |
| F_{X} | X pulling force on cantilever 10 due to membrane 20 extension |
| F_{Y} | Y pulling force on cantilever 10 due to membrane 20 extension |
| θ₁ | Rotation of cantilever 10's tip 13 (initial, without membrane 20) |
| δ₁ | Deflection of cantilever 10's tip 13 (initial, without membrane 20) |
| θ₁′ | Rotation of cantilever 10's tip 13 (with membrane 20) |
| δ₁′ | Deflection of cantilever 10's tip 13 (with membrane 20) |
| δ_{C} | Deflection of cantilever 10's tip 13 due to membrane 20 pulling force |
| θ₂ | Rotation of reflective panel portion 25 (on membrane 20) |
| δ₂ | Deflection of membrane 20 |
| K | Curvature of cantilever 10 |

Referring to FIGS. 2A to 2C, membrane 20 is attached to substrate material 8 at a first end 21 and attached to cantilever 10 at a second end 23. The length of membrane 20, measured from the edge of substrate material 8 to the edge of cantilever 10 as shown, is denoted by L₂. A reflective paddle portion 25 with a length Lₚ along the direction of the cantilever may be coupled with membrane 20 to facilitate optical detection of membrane rotation.

L₂ is less than L₁. However, the magnitude of the deflection of second end 23 of membrane 20 is about the same as the magnitude of the deflection of second end 13 of cantilever 10. Therefore, the reflective membrane 20 rotation, denoted by θ₂, is greater than the cantilever 10 rotation θ₁.

Since membrane 20 need not be configured to interact with its environment (e.g., to sense physical, biological, or chemical materials or interactions), its properties may be chosen to enhance rotation θ₂, and thus to increase the change in position of the reflected beam upon cantilever deflection. Therefore, the present sensor system provides enhanced optical sensitivity over existing sensors.

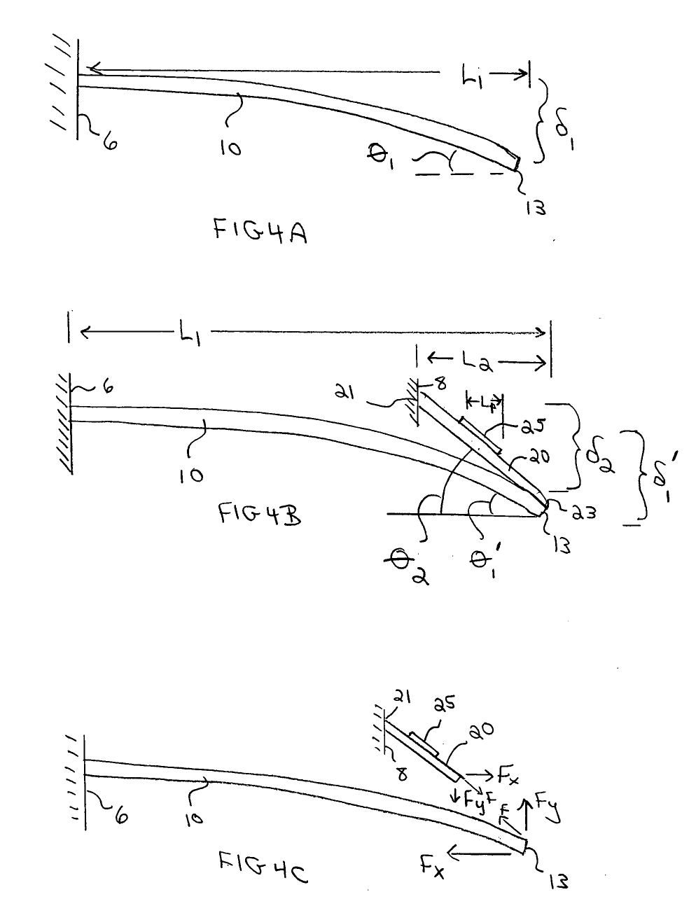

The reflective membrane rotation θ₂ may be determined by first analyzing the cantilever deflection without a membrane (see FIG. 4A), then using parameters from that analysis to determine the reflective membrane rotation (see FIG. 4B).

Equations 1, 2, and 3 below outline the relationship between the curvature (K), deflection (δ₁), and rotation (θ₁) of the cantilever tip 13 (without the reflective membrane 20 structure attached thereto), due to the induced surface stresses (γ). FIG. 4A shows a cantilever 10 with a cantilever tip angle θ₁ attached to a wall 6.

$$\begin{matrix}K = \frac{6 ( 1 - {v}_{1} ) γ}{{E}_{1} {t}_{1}^{2}} & Equation ( 1 ) \\ {δ}_{1} = \frac{1}{2} {KL}_{1}^{2} = \frac{3 γ ( 1 - {v}_{1} ) {L}_{1}^{2}}{{E}_{1} {t}_{1}^{2}} & Equation ( 2 ) \\ {θ}_{1} = {KL}_{1} = \frac{6 γ ( 1 - {v}_{1} ) {L}_{1}}{{E}_{1} {t}_{1}^{2}} & Equation ( 3 )\end{matrix}$$

The deflection of the cantilever tip 13 with the reflective membrane structure 20 attached thereto is determined using the superposition principle. FIG. 4B shows a cantilever 10 and a membrane 20 with a reflective portion 25. A first end 11 of cantilever 20 is attached to a fixed wall 6. A first end 21 of membrane 20 is attached to fixed substrate 8. A second end 23 of membrane 20 is attached to a second end 23 of cantilever 20.

In FIG. 4B, the deflection and rotation of cantilever 10 are denoted by δ₁′ and δ₁′, while the deflection and rotation of reflective membrane 20 are denoted by δ₂ and θ₂. The extension ΔL and strain ε of the reflective membrane as a result of δ₂ and θ₂ are given by Equations (4) and (5) below (approximately, for small angles θ₂):

$$\begin{matrix}Δ L = \frac{{L}_{2} {θ}_{2}^{2}}{2} & Equation ( 4 ) \\ ɛ = \frac{{L}_{2} {θ}_{2}^{2}}{2 {L}_{3}} & Equation ( 5 )\end{matrix}$$

Tensile force F in the reflective membrane due to this strain is given by Equation (6) below. This tensile force can be resolved into normal and tangential forces F_{X} and F_{Y}, which are given by Equations (7) and (8). The free body diagram of the composite structure is shown in FIG. 4C.

$$\begin{matrix}F = ɛ {E}_{2} {A}_{2} & Equation ( 6 ) \\ {F}_{X} = {ɛ}_{2} {E}_{2} {A}_{2} cos ( {θ}_{2} ) = \frac{{E}_{2} {w}_{2} {t}_{2} {L}_{2} {θ}_{2}^{2}}{2 {L}_{3}} & Equation ( 7 ) \\ {F}_{X} = {ɛ}_{2} {E}_{2} {A}_{2} sin ( {θ}_{2} ) = \frac{{E}_{2} {w}_{2} {t}_{2} {L}_{2} {θ}_{2}^{3}}{2 {L}_{3}} & Equation ( 8 )\end{matrix}$$

The deflection 62 and rotation θ₂ of the reflective membrane are related as shown in Equation (9) below. The deflection of the cantilever δ_{C} due to the force F_{Y} is given by Equation (10). The vertical force F_{Y} tries to bend the suspended cantilever in a direction opposite to δ₁ (see FIG. 4C). The final deflection of the cantilever δ₁′, which is the sum of the initial deflection δ₁ and δ_{C}, is given by Equation (11).

$$\begin{matrix}{δ}_{2} = {L}_{2} {θ}_{2} & Equation ( 9 ) \\ {δ}_{C} = - ( \frac{{E}_{2}}{{E}_{1}} ) ( \frac{{w}_{2}}{{w}_{1}} ) ( \frac{{L}_{2}}{{L}_{3}} ) \frac{2 {L}_{1}^{3} {θ}_{2}^{3} {t}_{2}}{{t}_{1}^{3}} & Equation ( 10 ) \\ {δ}_{1}^{′} = \frac{3 γ ( 1 - {v}_{1} ) {L}_{1}^{2}}{{E}_{1} {t}_{1}^{2}} - ( \frac{{E}_{2}}{{E}_{1}} ) ( \frac{{w}_{2}}{{w}_{1}} ) ( \frac{{L}_{2}}{{L}_{3}} ) \frac{2 {L}_{1}^{3} {θ}_{2}^{3} {t}_{2}}{{t}_{1}^{3}} & Equation ( 11 )\end{matrix}$$

The geometrical constraint on the system requires that the reflective membrane deflection δ₂ and cantilever deflection δ₁′ are the same and given by Equation (12) below:

$${L}_{2} {θ}_{2} = \frac{3 γ ( 1 - {v}_{1} ) {L}_{1}^{2}}{{E}_{1} {t}_{1}^{2}} - ( \frac{{E}_{2}}{{E}_{1}} ) ( \frac{{w}_{2}}{{w}_{1}} ) ( \frac{{L}_{2}}{{L}_{3}} ) \frac{2 {L}_{1}^{3} {θ}_{2}^{3} {t}_{2}}{{t}_{1}^{3}} \qquad Equation ( 12 )$$

The reflective membrane rotation θ₂ can be obtained by solving Equation (12).

FIG. 6 shows expected mechanical amplification versus membrane length L₃ for a silicon nitride cantilever and parylene membrane having the properties listed in Table 2 below.

TABLE 2

| L₁ | 500 μm |
|---|---|
| w₁ | 50 μm |
| t₁ | 0.6 μm |
| E₁ | 110 GPa |
| ν₁ | 0.25 |

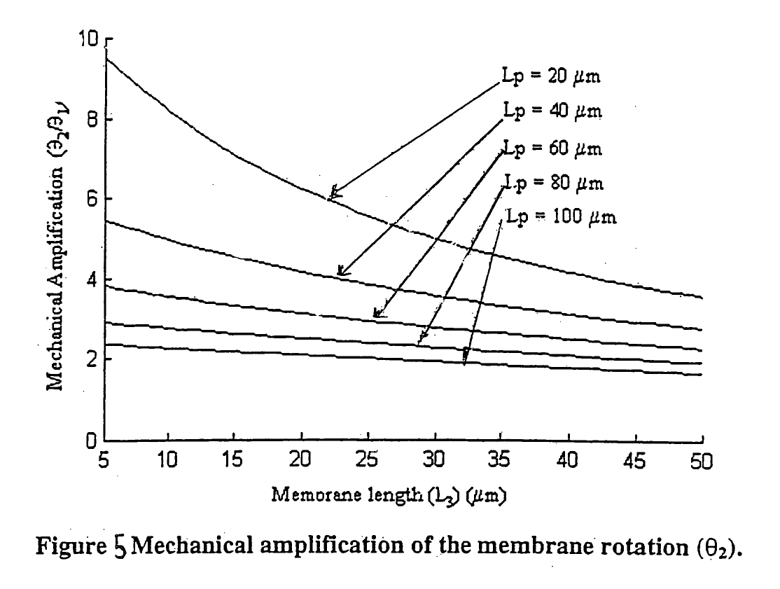

FIG. 5 shows that larger reflective paddle portion 25 lengths result in less mechanical amplification of the rotation. However, the size of the reflective paddle portion 25 should be large enough to reflect a spot that can be easily detected by a PSD or a CCD.

In accordance with various embodiments of the present invention, the microcantilever and micromembrane has reaction surfaces that are configured for any or all of: molecule to molecule, molecule to cell, cell to molecule and cell to cell bonding.

Specifically, in accordance with the present invention, the sensor (i.e. the cantilever or membrane) is not only configured not only for target-to-probe “molecule-to-molecule” bonding (in which bonding interactions occur between target molecules in a fluid or gas sample and probe molecules on the cantilever/membrane). Instead, the present cantilever (or membrane) is configured to be functionalized with either of “probe molecules” or “probe cells” attached thereto. Similarly, “target molecules” or “target cells” (in the fluid or gas sample) bond with the probe molecules or cells (on the cantilever/membrane). The resulting cantilever or membrane deflection is preferably detected so as to provide an indication of the reaction between the probe substance (which may be molecules or cells, or both) on the cantilever/membrane, and the target substance (which may be molecules or cells, or both) in the fluid or gas surrounding the cantilever/membrane. In accordance with the present invention, the probe substance is attached (i.e. functionalized) to the cantilever/membrane need not be a molecule. Rather, it may be either a molecule or a cell (or combinations of both). Moreover, in accordance with the present invention, the (target) substance need not be a molecule. Rather, it may be either a molecule or a cell (or combinations of both).

Thus, the present invention includes any or all of the following combinations of reactions:
(1) probe molecules interacting with target molecules;
(2) probe molecules interacting with target cells;
(3) probe cells interacting with target molecules; and
(4) probe cells interacting with target cells.

It is to be understood that the presently claimed probe/target “molecules” and “cells” are not limited to any particular “molecule” or “cell” per se. Rather, the present invention encompasses all inter-reactions found between or among various molecules and cells.

A number of implementations have been described. Nevertheless, it will be understood that various modifications may be made without departing from the spirit and scope of the invention. For example, the geometric and physical properties of the structure may be different. Different cantilever materials, membrane materials, lengths, widths, thicknesses, etc. may be used. Different fabrication processes may be used. Accordingly, other embodiments are within the scope of the following claims.

1. A sensor system, comprising:
a cantilever having a first end and a second end, the first end being held at a first fixed location and the second end being free to move; and
a reflective membrane having a first end and a second end, the first end being held at a second fixed location and the second end being attached to the cantilever.

2. The system of claim 1, wherein the reflective membrane comprises:
a layer of reflective material disposed on the reflective membrane.

3. The system of claim 1, wherein the reflective membrane comprises:
a panel of reflective material attached to the reflective membrane.

4. The system of claim 1, wherein a first portion of the reflective membrane is reflective, and a second portion of the reflective membrane is not reflective.

5. The system of claim 1, wherein the cantilever is longer than the reflective membrane.

6. The system of claim 1, further comprising:
a substrate, wherein the first end of the reflective membrane is attached to the substrate; and
a wall, wherein both the substrate and the cantilever are attached to the wall, and wherein the substrate and the cantilever extend in the same direction away from the wall.

7. The system of claim 6, wherein the substrate and the cantilever are generally parallel to one another prior to cantilever deflection.

8. The system of claim 1, wherein deflection of the cantilever causes rotation of the reflective membrane.

9. The system of claim 8, wherein the angle to which the reflective membrane rotates is greater than the angle to which the portion of the cantilever to which the second end of the reflective membrane is attached rotates.

10. The system of claim 1, wherein the angle of rotation of the reflective membrane is greater than the angle of rotation of the second end of the cantilever when the cantilever deflects.

11. The system of claim 1, wherein the reflecting membrane comprises:
a pair of membrane strips with a panel of reflective material disposed thereacross.

12. The system of claim 1, wherein the cantilever has probe molecules or cells disposed thereon.

13. The system of claim 12, wherein the cantilever deflects in response to target molecules or cells interacting with the probe molecules or cells.

14. A sensor system configured to amplify cantilever deflection, comprising:
a reflective membrane connected to a cantilever, wherein the reflective membrane rotates more than the cantilever when the cantilever deflects.

15. The sensor system of claim 14, wherein the reflective membrane rotates more than the portion of the cantilever connected to the reflective membrane when the cantilever deflects.

16. The system of claim 15, wherein the cantilever is longer than the reflective membrane.

17. The system of claim 16, wherein the cantilever has a first end and a second end, the first end being held at a fixed location and the second end being free to move, and wherein the reflective membrane has a first end and a second end, the first end being held at a fixed location and the second end being attached to the cantilever.

18. A method of sensing cantilever deflection, comprising:
directing a beam of incident light towards a reflective membrane, wherein a first end of the reflective membrane is held at a fixed location and a second end of the reflective membrane is attached to a cantilever;
deflecting the cantilever; and
detecting movement of a beam of light reflected by the reflective membrane.

19. The method of claim 18, wherein the cantilever is longer than the reflective membrane.

20. The method of claim 18, wherein deflecting the cantilever comprises:
exposing the cantilever to target molecules or cells, wherein the cantilever deflects in response to the target molecules or cells interacting with probe molecules or cells disposed on the cantilever.

21. The method of claim 20, wherein detecting the movement of the beam of light reflected by the reflective membrane corresponds to determining target molecule or cell concentration.

22. The method of claim 18, wherein the angle to which the reflective membrane rotates is greater than the angle to which the cantilever rotates.

23. The sensor system of claim 1, wherein the cantilever is stiffer than the reflective membrane.

24. A deflectable two-part sensor, comprising:
a first part being stiff which deflects in response to bonding thereon; and
a second part being compliant and optically reflective, wherein the first and second parts are connected together such that deflection of the first part results in amplification of the deflection of the second part.

25. The sensor of claim 24, wherein the first part is a functionalized cantilever, and the second part is a reflective membrane attached thereto.

26. The sensor of claim 24, wherein one end of each of the first and second parts of the sensor are supported at fixed locations.

27. The sensor of claim 24, wherein the first part of the sensor is longer than the second part of the sensor.

28. The sensor of claim 24, wherein the first and second parts are connected together such that rotation of the first part results in amplification of the rotation of the second part.
